(19) United States
(12) Patent Application Publication
(10) Pub. No.: US 2012/0123549 A1
(43) Pub. Date: May 17, 2012

(54) Jawbone Prosthesis and Method of Manufacture

(75) Inventors: Guo-Feng Xu, Guangzhou (CN); Bin Xu, Guangzhou (CN)

(73) Assignee: GRANDHOPE BIOTECH CO., LTD., Guangzhou (CN)

(21) Appl. No.: 13/360,256

(22) Filed: Jan. 27, 2012

Related U.S. Application Data
(63) Continuation of application No. 12/288,924, filed on Oct. 24, 2008.
Application No. 12/288,924 is a continuation-in-part of application No. 11/494,817, filed on Jul. 28, 2006.

(51) Int. Cl. A61F 2/02

(52) U.S. Cl. 623/17.17

(57) ABSTRACT

A biological jawbone prosthesis is made according to a method that includes the steps of collecting animal material from a bovine or porcine source, the animal material being a jawbone, shaping the animal material to provide a desired shape for the jawbone implant, removing cells from the animal material, crosslinking the animal material, removing antigens from the animal material, subjecting the animal material to an alkaline treatment, coupling into the animal material active substances which are capable of adhering growth factor and stem cell, and packing the animal material in a container that contains a sterilization solution.

### BACKGROUND OF THE INVENTION

Related Cases

This is a continuation application of co-pending Ser. No. 12/288,924, filed Oct. 24, 2008, and a continuation-in-part of co-pending Ser. No. 11/494,817, filed Jul. 28, 2006, whose disclosures are incorporated by this reference as though fully set forth herein.

1. Field of the Invention

The present invention relates to a medical prosthesis for human implantation, and in particular, to a jawbone prosthesis used in cosmetic surgery of the jawbone or in the repair of the jawbone.

2. Description of the Prior Art

Reconditioning and elongation of the jawbone are often performed in cosmetic and prosthetic surgeries. Implants called artificial or prosthetic jawbones are typically utilized in these surgeries. All such implants are currently produced using synthetic materials such as silicone rubber or PTFE. Unfortunately, these implants of synthetic materials have nothing in common with the human jawbone in terms of composition and structure. Displacement, wear, puncture and exposure due to erosion could occur to such implants after a long period of time because, even if they can peacefully coexist within the body, they are foreign matter and are incompatible with the host tissue. In addition, these synthetic materials could also cause irritating discomfort to the patient.

Thus, there still remains a need for a biological jawbone prosthesis which avoids the drawbacks described above.

### SUMMARY OF THE DISCLOSURE

In order to accomplish the objects of the present invention, the present invention provides a biological jawbone prosthesis made according to a method that comprises the following steps:

collecting animal material from a bovine or porcine source, the animal material being a jawbone;

### BACKGROUND OF THE INVENTION

1. Field of the Invention

The present invention relates to a medical prosthesis for human implantation, and in particular, to a jawbone prosthesis used in cosmetic surgery of the jawbone or in the repair of the jawbone.

2. Description of the Prior Art

Reconditioning and elongation of the jawbone are often performed in cosmetic and prosthetic surgeries. Implants called artificial or prosthetic jawbones are typically utilized in these surgeries. All such implants are currently produced using synthetic materials such as silicone rubber or PTFE. Unfortunately, these implants of synthetic materials have nothing in common with the human jawbone in terms of composition and structure. Displacement, wear, puncture and exposure due to erosion could occur to such implants after a long period of time because, even if they can peacefully coexist within the body, they are foreign matter and are incompatible with the host tissue. In addition, these synthetic materials could also cause irritating discomfort to the patient.

Thus, there still remains a need for a biological jawbone prosthesis which avoids the drawbacks described above.

### SUMMARY OF THE DISCLOSURE

In order to accomplish the objects of the present invention, the present invention provides a biological jawbone prosthesis made according to a method that comprises the following steps:

collecting animal material from a bovine or porcine source, the animal material being a jawbone;

shaping the animal material to provide a desired shape for the jawbone implant;

removing cells from the animal material;

crosslinking the animal material;

removing antigens from the animal material;

subjecting the animal material to an alkaline treatment;

coupling into the animal material active substances which are capable of adhering growth factor and stem cell; and

packing the animal material in a container that contains a sterilization solution.

The biological artificial jawbone of the present invention has no immune rejection and has excellent tissue compatibility after being treated by multiform antigen removal processes, and with tissue induction techniques, because the composition and structure are similar to those of natural bone. The prosthesis of the present invention can coexist with the host jawbone tissues for a long period of time after being implanted and becomes part of the host jawbone, creating none of the irritating discomfort of foreign matter and having no drawbacks such as displacement, wear or exposure.

### BRIEF DESCRIPTION OF THE DRAWINGS

FIG. 1 is an oblique view from the horizontal direction of a biological jawbone prosthesis according to one embodiment of the present invention.

FIG. 2 is a front view in the vertical direction of the prosthesis of FIG. 1.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

The present invention provides a method for producing a biological artificial jawbone that utilizes animal bone as the material. The raw material is first purified and processed, the cells are removed, and then the material is fixed using epoxide. Thereafter, multifold antigen removal technology, tissue induction technology, and a series of other biochemical technological processes are applied. The prosthesis is rinsed and packaged. High permeation techniques are employed for important biochemical treatments in order for the treatment reagents to penetrate and exert their effects deep inside the microcavities of the bone tissue. A high permeation reactor which is driven by combined movements of ultrasonic vibration and vacuum pulse is used to allow the treatment reagents to exert the effects in the high permeation reactor. The specific technical workflow process for preparation is as follows:

1. Pretreatment (sterilization and removal of foreign matter)

2. Mechanical processing/molding

3. Cell removal

4. Crosslinking and fixation

4a. NaOH Treatment (only if from a bovine source)

5. Antigen removal

6. Technical treatment of tissue induction

7. Rinsing

8. Sterilization, Sealing and packaging

Step 1: In the pretreatment step, the jawbone material is collected from bovine or porcine sources using techniques that are well-known in the art. The bone material is immersed and sterilized in broad-spectrum antibacterial agents, and impurities such as bone membranes are removed by stripping them off using techniques that are well-known in the art.

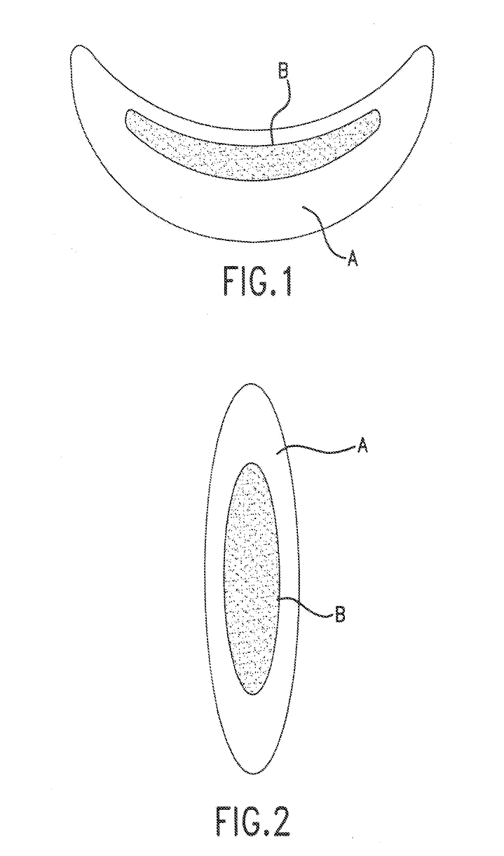

Step 2: In the mechanical processing/molding step, the bone material is processed using well-known tools and methods into the desired shape, such as that shown in FIG. 1, which is preferably a shape that conforms to the human jawbone.

Step 3: In the cell removal step, cells (i.e., all types of cells present in the bone material) are removed by enzymolysis and/or by washing with a cleansing agent (surfactant). The enzyme utilized in the enzymolysis can be pepsin or trypsin. Examples of the surfactant utilized as the cleansing agent for the washing treatment can include Tween-20, emulsifier OP-10 and Triton X-100.

Step 4: The crosslinking and fixation step involves carrying out a crosslinking reaction between an epoxide utilized as the crosslinking-fixation agent and the organic base substances in the artificial bone. The reaction can be conducted at 5-50° C. for 8 to 96 hours, and the epoxide can be selected from the following substances:

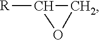

R═CₙH₂ₙ₊₁-group or

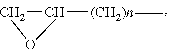

wherein n is selected from 0, 1, 2 . . . 12. The reagent concentration is 0.1-1.5N.

Step 5: According to modern immunological theory, the antigenicity of animal tissues stems mainly from active groups located at specific sites and in specific conformations, and these active groups include —H₂*,—OH*, —SH*, etc. The specific conformations result mainly from some specific hydrogen bonding formed by spiral protein chains. The specific sites and conformations are called antigen determinants. The antigen removal step uses multiple reagents to block the active groups and alter the special conformation. The reagents used to block specific active groups are mainly nucleophilic reagents that react easily with —H₂*,—OH*, —SH* and other similar groups. These reagents include carboxylic acid anhydrides, acyl chlorides, acylamides, epoxy compounds, etc. The reagents that can be used to alter specific conformations include class one strong hydrogen bond formation agents, such as guanidine hydrochloride. Because the specific conformations result mainly from some specific hydrogen bonding formed by spiral protein chains, using strong hydrogen bond formation agents to replace the specific hydrogen bond makes it possible to change the specific conformation. Here the * symbol on the groups indicates that they are a small number of specific groups which are located in specific locations and are able to produce a response to immune signals, and they are not the standard —NH₂, —OH, —SH groups. These specific groups are in a high-energy activity state. preferable for nucleophilic reagent initiated reactions, just as the catalyst's active center is preferable for the reactant or toxin reaction.

Step 6: The technical treatment of tissue induction involves coupling an active substance capable of adhering growth factors or stem cells to facilitate the accumulation of growth factors and stem cells released by the self-repair mechanism of the body on the implant and delivering them to the wound area, while facilitating high expression for a long period of time and promoting the assimilation of the artificial jawbone and the host jawbone. The active substances introduced can include some specific polypeptides or glycosaminoglycan compounds. The main specific polypeptides are mainly polypeptides consisting of oligopeptides of 16 lysines and arginine, glycine and aspartic acid such as Lys (16)-Gly-Arg-Asp-Ser-Pro-Cys; examples of the glycosaminoglycans include hyaluronic acid, chondroitin sulfate, dermatan sulfate, keratin sulfate, heparin and acetylheparin sulfate. The method of introduction may be accomplished by coupling, chemical adsorption, physical adsorption, or collagen membrane inclusion. Coupling is preferred, and coupling agents that may be used include difunctional compounds such as dicarboxylic acid anhydrides, diacyl diamines, diacyl dichlorides, diepoxides and carbodiimides.

Step 7: Rinsing involves rinsing off excessive chemical or bio-agents with purified water.

Step 8: In the sterilization, sealing and packaging step, the prosthesis is sealed in a dual-layer plastic bag containing physiological saline storage solution. The packed product can then sterilized under minimum 25 kGy γ-irradiation. This sterilization method has been proven to kill known pathogens, except prions.

Step 4a: An additional “NaOH treatment” step is required between the crosslinking-fixation treatment and the multiform removal of antigens if the bone material is from a bovine source. In this step, the article is immersed in 1N NaOH at 25-50° C. for more than 60 min to kill prion viruses that may be present.

Steps 3-7 in the aforementioned treatment processes can be performed in a high permeation reactor. The reactor can be an air-tight vessel furnished with an ultrasonic vibrating device and a vacuum pulse device. Vacuum pulse can be used to remove air inside the bone material, and when used-in combination ultrasonic vibration, the reagents can permeate the micropores deep inside the bone material to ensure that the material is thoroughly treated with all the necessary reagents. In this regard, all the treatments in steps 3-7 can be carried out in the same reactor, though different reagents may be used in the different steps.

The superiority of the biological artificial jawbone of the present invention over the conventional prosthetic jawbones made of silicone rubber or PTFE rests on the fact that the biological artificial jawbone is produced from pure natural materials, and that the composition and structure are similar to those of the natural human jawbone, thereby having excellent biocompatibility, while causing no immune rejection. As a result, the prosthesis can grow into the host jawbone tissue and the two can assimilate into one body. The drawbacks experienced by the conventional prosthetic jawbones, such as persistent irritation from foreign matter, displacement, wear, puncture of skin tissue or exposure due to erosion, can therefore be avoided.

### EXAMPLE

Fresh healthy porcine bone is screened and sterilized by immersion in 0.1% benzalkonium bromide. The bone membrane is removed after the bone is taken out, followed by processing and molding into the shape shown in FIG. 1 using a special tool, which is cleaned. The article is placed in a high permeation reactor and 40-200 mg/L pepsin or trypsin is added to carry out enzymolysis at 18-45° C. for 2-16 h. The article is then placed in a high permeation reactor after the enzyme is eluted and deactivated, and 0.1-2N epoxide is added for reaction at 5-40° C. for 8-96 h. The epoxide is selected from the following substances:

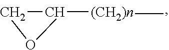

R═CₙH₂ₙ₊₁-group or

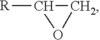

wherein n is selected from 0, 1 . . . 12. The epoxide is then neutralized and the article is washed, followed by conducting the antigen-removal reaction at 5-50° C. for 2-24 h in a high permeation reactor (which can be the same reactor as above) by adding antigen-removal agents. Examples of the antigen-removal agents utilized include carboxylic anhydrides, acyl chlorides, epoxides and guanidine hydrochloride. Two or more antigen-removal agents are utilized for the reaction in order to ensure complete removal of the antigens. The article is washed and reacted at 5-30° C. for 2-24 h in a high permeation reactor (which can be the same reactor as above) by adding two active substances, namely a polypeptide consisting of Lys (16)-Gly-Arg-Asp-Ser-Pro-Cys and the coupling agent glutaric acid anhydride. The article is washed, packaged and sealed, followed by sterilization, by irradiation to yield the final product.

While the description above refers to particular embodiments of the present invention, it will be understood that many modifications may be made without departing from the spirit thereof. The accompanying claims are intended to cover such modifications as would fall within the true scope and spirit of the present invention.

1-14. (canceled)

15. A jawbone implant made according to a method that comprises the following steps:
isolating from a host a natural animal jawbone that has a substrate;
shaping the animal material to provide a desired shape for the jawbone implant;
removing cells from the animal material;
crosslinking and fixing the animal material;
blocking residual specific active groups in protein molecules of the animal material after fixation by applying at least one active reagent;
altering the specific conformation of protein molecules of the animal material by a reagent with strong hydrogen bonding power;
subjecting the animal material to an alkaline treatment;
coupling into the animal material active substances which are capable of adhering growth factor and stem cell; and
packing the animal material in a container that contains a sterilization solution.

16. The implant of claim 15, wherein the cell removal step uses enzymolysis and/or washing with a surfactant.

17. The implant of claim 15, wherein the crosslinking step is implemented using the epoxy compound 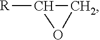
R═CₙH₂ₙ₊₁ group or 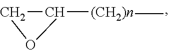
n 32 0, 1, 2, 3 . . . 12, as the crosslinking agent.

18. The implant of claim 15, wherein the active substances are polypeptides containing 16 lysine oligopeptides with arginine, glycine, and aspartic acid.

19. The implant of claim 15, wherein the cell removal step uses an enzymatic method or a detergent elution method to remove cells.

20. The implant of claim 19, wherein the enzymatic method uses trypsin or pepsin to perform enzymatic action.

21. The implant of claim 15, wherein the at least one active reagent to block specific active groups in the protein molecules of the substrate can be acid anhydrides, acid chlorides, or acylamides.

22. The implant of claim 15, wherein the reagent with strong hydrogen bonding power is a guanidine compound.

23. The implant of claim 15, wherein the alkaline treatment step uses 1-4N sodium hydroxide to immerse the animal material for a fixed period of time.

24. The implant of claim 15, wherein the animal material is fixed by an epoxy compound that has a hydrocarbon backbone, that is water-soluble, and which does not contain an ether or ester linkage in its backbone.
